(19) United States
(12) Patent Application Publication
(10) Pub. No.: US 2018/0348094 A1
(43) Pub. Date: Dec. 6, 2018

(54) GAS DETECTION INSTRUMENT INLET NOZZLE ASSEMBLY

(71) Applicant: Gas Measurement Instruments Limited, St. Paul, MN (US)

(72) Inventors: Andrew Corr, Glasgow (GB); George McGregor, Glasgow (GB)

(21) Appl. No.: 15/771,507

(22) PCT Filed: Oct. 20, 2016
(86) PCT No.: PCT/GB2016/053280

§ 371 Date: Apr. 27, 2018

(30) Foreign Application Priority Data
Oct. 29, 2015 (GB) 1519143.0

(51) Int. Cl. G01N 1/22; G01N 33/00

(52) U.S. Cl. CPC G01N 1/2205; G01N 33/0014

(57) ABSTRACT

A gas inlet nozzle assembly comprises an inlet tube (10) having an inlet end (10a) and an outlet end (10b). A hydrophobic filter (15) is removably engageable in the inlet tube (10) through the inlet end (10a), and a particulate filter (12) is removably engageable in the inlet tube (10) through the inlet end (10a). A retainer (13) is lockingly engageable in the inlet end (10a) of the inlet tube (10) so as to retain the hydrophobic filter (15) and the particulate filter (12) in the inlet tube (10).

The present invention relates to gas detection instruments and more particular to inlet nozzle assemblies which form part of such gas detection instruments.

Typical gas detection instruments include a particulate filter and a hydrophobic filter in the gas inlet to help to protect the instrument from damage from moisture and foreign objects by preventing particles such as dust, and water from being drawn into the instrument by the internal pump and damaging the sensors. Due to the natural of the practical field use of these type of instruments, and because the filters are the first line of defence for the instruments, the filters are considered to be serviceable parts which will need to be replaced numerous times during the operating life of the instrument.

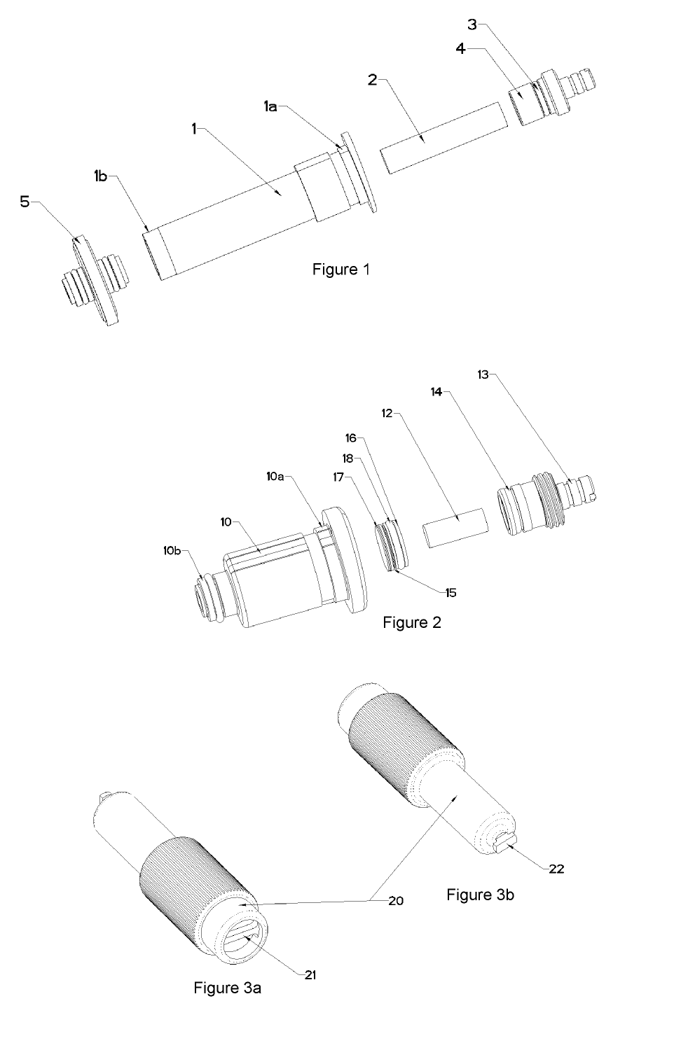

In designs known in the art, as exemplified by FIG. 1, the particulate filter is integrated into the inlet assembly so that it can easily be removed and replaced externally of the instrument without having to dis-assemble the whole instrument, and the particulate filter can therefore easily be removed and replaced by the user in the field when required.

However, conventional design places the hydrophobic filter internally of the instrument, downstream of the inlet assembly. As a result, in order to remove and replace this filter, the whole instrument must be dis-assembled, a job which cannot reasonably be done in the field and normally necessitates returning the instrument to the manufacturer or a service centre. This is not only costly to the user but also means the user is without the instrument during the time taken to complete the service.

According to the present invention there is provided a gas inlet nozzle assembly comprising an inlet tube have an inlet end and an outlet end, a hydrophobic filter removably engageable in the inlet tube through the inlet end, a particulate filter removably engageable in the inlet tube through the inlet end, and a retainer lockingly engageable in the inlet end of the inlet tube so as to retain the hydrophobic filter and the particulate filter in the inlet tube.

A gas inlet nozzle assembly in accordance with the present invention has the advantage that by having both the hydrophobic filter and the particulate filter removably mounted in the inlet tube through the inlet end, bot filters can be removed and replaced without necessitating removal of the inlet tube and hence without requiring disassembly of the instrument. As a result, both filters can be serviced without requiring return to the manufacturer, and instead field replacement of the filters can easily be carried out by the user.

Preferably, the retainer is formed separately of the particulate filter and engages in the inlet tube upstream of the hydrophobic and particular filters. However, retainer may instead be integrated with the particular filter. The retainer furthermore preferably includes locking means which engage with complimentary locking means formed on the inlet tube, such as male and female threads, to locking secure the retainer to the inlet tube.

The retainer furthermore preferably includes seal means such as an O ring which engages with the inlet tube for effecting a fluid tight seal between the retainer and the inlet tube.

The inlet tube preferably includes a seat on its inner surface proximate the outlet end against which the hydrophobic filter engages. The hydrophobic filter further preferably includes seal means which engages with the inlet tube to effect a fluid tight seal between the hydrophobic filter and the inlet tube and thereby constrains fluid passing through the inlet assemble to pass through the hydrophobic filter. In particular, the seal means may engage with the seat of the inlet tube, but preferably the hydrophobic filter is mounted in a filter housing so as to form a hydrophobic filter assembly, a seal such as an O ring seal being provided on the outer surface of the filter housing which sealingly engages with the inner surface of the inlet tube to effect a fluid tight seal therebetween and thereby prevent fluid from passing between the housing and the inlet tube.

Preferably, the hydrophobic filter includes latching means which are engaged by complementary latching means provided on a removal tool for facilitating insertion and removal of the filter from the inlet tube. IN one embodiment, the latching means comprises a slotted opening provided in the hydrophobic filter, in particular in the filter housing, which is engaged by a spigot on the removal tool in a bayonet manner.

The present invention further provides a gas detection instrument comprising a housing containing a sensor, an inlet, a pump for delivering gas from the inlet to the sensor, and a gas inlet nozzle assembly according to the invention provided in the fluid flow path between the inlet and the pump.

IN order that the invention may be well understood, there will now be described an embodiment thereof, given by way of example, reference being made to the accompanying drawings, in which:

FIG. 1 is an exploded view of an inlet nozzle assembly according to the prior art;

FIG. 2 is an exploded view of an inlet nozzle assembly according to the invention; and

FIGS. 3a and 3b are perspective views of a removal tool suitable for use with the inlet nozzle assembly of the invention.

Referring first to FIG. 1, there is show a conventional inlet nozzle assembly known in the art. The assembly comprises an inlet tube 1 having an inlet end 1a, an outlet end 1b, and a tubular opening connecting the inlet end 1a to the outlet end 1b. A cylindrical dust filter 2 is removably engageable in the tubular opening of the inlet tube 1 from the inlet end 1a thereof, and is retained in place by an inlet connector 3 which is lockingly engageable in the inlet end 1a. The inlet connector 3 includes an o-ring seal 4 for effecting a sealing engagement with the tubular opening of the inlet tube 1.

A hydrophobic filter 5 is sealingly connected to the outlet end 1b of the inlet tube such that fluid passing through the inlet tube 1 is constrained to pass through the hydrophobic filter 5. The hydrophobic filter 5 has a connector on both sides for connecting to the inlet tube and for connecting to conduit for connection with a pump of a gas instrument (not shown). Due to the size and location of the hydrophobic filter 5, the inlet tube 1 must be removed in order to gain access to the hydrophobic filter for servicing and/or removal.

Referring now to FIG. 2, there is shown an inlet nozzle assembly according to the invention.

The assembly comprises an inlet tube 10 having an inlet end 10a, an outlet end 10b and a tubular opening connecting the inlet 10a to the outlet 10b. The outlet end 10b is formed as a connector for connecting the inlet tube 10 to a pump of a gas detection instrument.

The inlet nozzle 10 has a shoulder proximate its outlet end 10b which forms a seat in the tubular opening against which is engageable a hydrophobic filter assembly 15, which is insertable into the tubular opening through the inlet end 10a of the inlet tube. The hydrophobic filter assembly 15 is formed by a filter housing 16 on which is mounted a hydrophobic filter PTFE membrane 17. In the preferred embodiment, the membrane 17 has a pore size of 5 micro-meters. An o-ring seal 18 is mounted around the outside of the housing 16 which sealingly engages the surface of the tubular opening of the inlet tube 10 so as to prevent fluid passing around the outside of the filter housing 16 and thereby ensuring that all fluid passes through the hydrophobic filter 17,

A particulate filter 12 is also engageable in the tubular opening of the inlet tube behind the hydrophobic filter assembly 15 such that all fluid passes through the particulate filter 12 before reaching the hydrophobic filter 17. The particulate filter 12 is sized to engage in the end of an inlet connector 13 which is lockingly engageable in the inlet end 10a of the inlet tube so as to retain the hydrophobic filter assembly 15 and the particulate filter 12 in the inlet tube 10. In the illustrated embodiment, the inlet connector 13 has a male thread formed on its outer surface which screwingly engages with complementary female threads formed on the inner surface of the inlet tube 10 proximate the inlet end 10a therefore, but other coupling means are also possible within the scope of the invention. An O-ring seal 14 is carried on the outer surface of the inlet connector 13 for effecting a sealing engagement with the inner walls of the inlet tube 10 so as to ensure that all fluid entering the inlet tube does so through the inlet connector and is hence directed through the particulate filter 12 and subsequently the hydrophobic filter 17.

In order, then, to remove both the particulate filter 12 and the hydrophobic filter assembly 15, the user simply removes the inlet connector 13 by unscrewing it from the inlet tube 10. To facilitate such removal, the inlet connector 13 is provided with a slotted end and a removal tool 20 is provided having a removal blade 21 on one end which is engageable with the slot in the inlet connector.

Upon removal of the inlet connector 13, the particulate filter 12, which is engaged in the inside end of the inlet connector 13, is also removed from the inlet tube 10 and can then easily be pulled out of the inlet connector 13 and serviced or replaced. The hydrophobic filter assembly 15 can also then be removed from the inlet tube 10 through the inlet end 10a thereof. Due to the close fit of the seal 18 with the internal surface of the tubular opening of the inlet tube 10, the hydrophobic filter assembly 15 will be a tight fit in the inlet tube 10 and not easily removed therefrom. In order, then, to facilitate such removal, the filter housing 16 is provided with a slotted opening in its end which faces the inlet end 10a of the inlet tube 10, and the removal tool 20 has a spigot 22 formed on its second end which is of complementary size to the slotted opening of the filter housing 16 such that the spigot 22 is engageable through the slotted opening and can then be rotated in a bayonet fashion to lock the removal tool 20 to the hydrophobic filter assembly 15. Once so locked, the removal tool 20 can then be used to withdraw the hydrophobic filter assembly 15 from the inlet tube 10 for servicing or replacement of the hydrophobic filter assembly 15. The removal tool 20 may similarly be used to facilitate replacement of a hydrophobic filter assembly 15 in place proximate the outlet end 10b of the inlet tube 10 without causing damage to the hydrophobic filter 17.

It will, of course, be understood that other designs of tool are also possible within the scope of the invention.

1. A gas inlet nozzle assembly comprising an inlet tube having an inlet end and an outlet end, a hydrophobic filter removably engageable in the inlet tube through the inlet end, a particulate filter removably engageable in the inlet tube through the inlet end, and a retainer lockingly engageable in the inlet end of the inlet tube so as to retain the hydrophobic filter and the particulate filter in the inlet tube.

2. A gas inlet nozzle assembly according to claim 1, wherein the retainer is formed separately of the particulate filter and engages in the inlet tube upstream of the hydrophobic and particular filters.

3. A gas inlet nozzle assembly according to claim 1, wherein the retainer is integrated with the particular filter.

4. A gas inlet nozzle assembly according to claim 1, wherein the retainer includes locking means which engage with complimentary locking means formed on the inlet tube to locking secure the retainer to the inlet tube.

5. A gas inlet nozzle assembly according to claim 1, wherein the retainer includes seal means which engages with the inlet tube for effecting a fluid tight seal between the retainer and the inlet tube.

6. A gas inlet nozzle assembly according to claim 5, wherein the seal is an O ring.

7. A gas inlet nozzle assembly according to claim 1, wherein the inlet tube includes a seat on its inner surface proximate the outlet end against which the hydrophobic filter engages.

8. A gas inlet nozzle assembly according to claim 7, wherein the hydrophobic filter includes seal means which engages with the inlet tube to effect a fluid tight seal between the hydrophobic filter and the inlet tube and thereby constrains fluid passing through the inlet assemble to pass through the hydrophobic filter.

9. A gas inlet nozzle assembly according to claim 8, wherein the seal means of the hydrophobic filter engages with the seat of the inlet tube.

10. A gas inlet nozzle assembly according to claim 8, wherein the hydrophobic filter is mounted in a filter housing so as to form a hydrophobic filter assembly, a seal being provided on the outer surface of the filter housing which sealingly engages with the inner surface of the inlet tube to effect a fluid tight seal therebetween and thereby prevent fluid from passing between the housing and the inlet tube.

11. A gas inlet nozzle assembly according to claim 9, wherein the seal is an O ring seal.

12. A gas inlet nozzle assembly according to claim 1, wherein the hydrophobic filter includes latching means which are engagable by complementary latching means provided on a removal tool for facilitating insertion and removal of the filter from the inlet tube.

13. A gas inlet nozzle assembly according to claim 12, wherein the latching means comprises a slotted opening provided in the hydrophobic filter which is engagable by a spigot on the removal tool in a bayonet manner.

14. A gas detection instrument comprising a housing containing a sensor, an inlet, a pump for delivering gas from the inlet to the sensor, and a gas inlet nozzle assembly according claim 1 provided in the fluid flow path between the inlet and the pump.

15. A gas detection instrument according to claim 14, further comprising a removal tool having latching means provided thereon which are complementary to latching means on the hydrophobic filter such that the removal tool is engageable with the hydrophobic filter to facilitate insertion and removal of the filter from the inlet tube.

16. A gas detection instrument according to claim 15, wherein the latching means in the hydrophobic filter comprises a slotted opening and the latching means on the removal tool is a spigot which engages with the slotted opening in a bayonet manner.

17. A gas detection instrument according to claim 15, wherein the removal tool includes a removal blade which is engageable with a slot in the inlet connector to facilitate removal of the inlet connector.
